(19) United States
(12) Patent Application Publication
(10) Pub. No.: US 2015/0133472 A1
(43) Pub. Date: May 14, 2015

(54) PHARMACEUTICAL COMPOSITION

(71) Applicant: CIPLA LIMITED, Mumbai (IN)

(72) Inventors: Shrinivas Purandare, Mumbai (IN); Geena Malhotra, Mumbai (IN)

(21) Appl. No.: 14/398,781

(22) PCT Filed: May 10, 2013
(86) PCT No.: PCT/GB2013/000211

§ 371 Date: Nov. 4, 2014

(30) Foreign Application Priority Data
May 11, 2012 (IN) 1444/MUM/2012

(51) Int. Cl. A61K 31/506; A61K 47/14; A61K 9/00; A61K 47/06; A61K 47/02; A61K 47/18; A61K 47/38; A61K 47/44; A61K 47/34

(52) U.S. Cl. CPC A61K 31/506; A61K 47/44; A61K 47/14; A61K 9/0048; A61K 47/34; A61K 47/02; A61K 47/186; A61K 47/38; A61K 47/06
USPC 514/256

(57) ABSTRACT

The present invention relates to a pharmaceutical composition comprising voriconazole and an aqueous, non-aqueous, or oily vehicle, or a mixture thereof, and optionally one or more pharmaceutically acceptable excipients; to a process for preparing such a composition, and to the use of such a composition for the prevention or treatment of fungal infections.

### FIELD OF THE INVENTION

The present invention relates to a pharmaceutical composition of voriconazole, to a process for preparing such a composition, and to therapeutic uses and a method of treatment employing the same.

### BACKGROUND AND PRIOR ART

Voriconazole, chemically designated as (2R,3S)-2-(2,4-Difluorophenyl)-3-(5-fluoro-4-pyrimidinyl)-1-(1H-1,2,4-triazol-1-yl)-butan-2-ol, is indicated for the treatment of various fungal infections caused by Aspergillus fumigatus and Aspergillus other than A. fumigatus, Candidemia, Esophageal candidiasis and serious fungal infections caused by Scedosporium apiospermum. It has the following chemical structure:

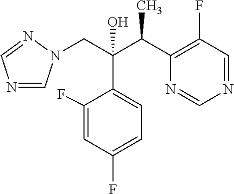 [SMILES: C[C@@H](c1ncncc1F)[C@](O)(Cn1cncn1)c1ccc(F)cc1F]

Voriconazole is disclosed in European patent EPO440372. U.S. Pat. Nos. 5,116,844; 5,364,938; 5,567,817; 5,773,443 and 6,632,803 describe voriconazole and its formulations.

Voriconazole has a low aqueous solubility (0.61 mg/ml at a pH 7; 0.2 mg/ml at a pH 3), and is not stable in water (an inactive enantiomer is formed from combination of the retro-aldol products of hydrolysis). Degradation of voriconazole occurs in aqueous solution, particularly under basic conditions. Thus, development of an aqueous formulation with a sufficient shelf life is difficult. These problems are further magnified by the semipolar nature of the compound (log D=1.8) which means that it is not generally solubilized by conventional means such as oils, surfactants or water miscible co-solvents. Additionally, voriconazole is not stable in water leading to increase in impurity. Therefore, development of voriconazole compositions generally with a controlled impurity profile poses a considerable developmental challenge.

In the lyophilized formulation of voriconazole for injection which is marketed by Pfizer Co. under the trade name Vfend®, the solubility of voriconazole is increased by using a solubilizer, sulfobutyl ether. β-cyclodextrin sodium (SBECD). The amount of SBECD in 1 milligram of lyophilized formulation of approved voriconazole (labeled amount) is about 15 mg to 18 mg (1:15). To increase the solubility of the drug, a large amount of SBECD has to be used in the lyophilized formulation.

Accordingly, various attempts have been made in the prior art to improve the stability of voriconazole in formulations.

European Patent EPO440372 discloses co-formulation with cyclodextrin derivatives to improve solubility; however, it is always desirable to keep the number of ingredients in a formulation to a minimum so as to minimize possible adverse reactions in patients. Further, underivatised or unmetabolised cyclodextrin may have toxic effects on the body and so may be unsuitable as a pharmaceutical excipient.

WO 98/58677 discloses that the solubility of voriconazole in water can be increased by molecular encapsulation with suiphoalkylether cyclodextrin derivatives of the type disclosed in WO 91/11172, particularly beta-cyclodextrin derivatives wherein the cyclodextrin ring is substituted by suiphobutyl groups. However, the said cyclodextrin encapsulated voriconazole may not remain stable when developed into aqueous ready-to-use compositions. Moreover, there are complex manufacturing issues associated with cyclodextrin formulations which also increase manufacturing cost significantly.

WO97/28169 discloses a phosphate pro-drug of voriconazole, which exhibits increased solubility and aqueous stability. However, the pro-drug may not exhibit 100% bioequivalence to voriconazole.

US2005112204 discloses a pharmaceutical formulation of voriconazole, in particular an aqueous micellar poloxamer preparation comprising voriconazole, and one or more poloxamer. The pharmaceutical acceptability of various poloxamers is well established, with certain species approved for parenteral administration. However, there have been problems with targeting and dispensing drugs using poloxamers. Munish et al., [cancer letters, 118(1997), 13-19] found that in some cases it was not possible for the drug to release, unless ultrasound was used to disrupt the micelles. The requirement of the use of ultrasound is expensive and undesirable.

Thus, as can be noted herein above there is insufficient disclosure about how to formulate a stable ready-to-use voriconazole composition. Hence there still exists a need to develop pharmaceutical compositions of voriconazole having improved stability over the storage period when formulated in the form of ready-to-use composition. There is also a need for suitable/stable voriconazole compositions that exhibit a controlled impurity profile.

Despite the above-noted inherent difficulties associated with formulating voriconazole, we have surprisingly found that stable compositions of the drug may be prepared with a variety of aqueous, non-aqueous, or oily vehicle, or mixtures thereof.

### OBJECT OF THE INVENTION

An object of the present invention is to provide a ready-to-use pharmaceutical composition of voriconazole having improved stability.

Another object of the present invention is to provide a ready to use ready-to-use pharmaceutical composition of voriconazole having improved stability and exhibiting controlled impurity profile.

Yet another object of the present invention is to provide a process for preparing a ready-to-use pharmaceutical composition comprising voriconazole having improved stability with the ease of manufacturing.

A further object of the present invention is to provide a method for prophylaxis or treatment of patients in need thereof which comprises administering a ready-to-use pharmaceutical composition comprising voriconazole having improved stability.

Still another object of the present invention is to provide the use of a ready-to-use pharmaceutical composition comprising voriconazole having improved stability for preventing or treating a topical or systemic fungal infection.

### SUMMARY OF THE INVENTION

According to one aspect of the invention, there is provided a stable composition comprising voriconazole or its salt, solvate, ester, derivatives, hydrate, enantiomer, polymorph, prodrugs, complex or mixtures thereof wherein the said drug is dispersed in an aqueous, non-aqueous, or oily vehicle, or mixtures thereof.

According to a second aspect of the present invention, there is provided a process for preparing a ready-to-use pharmaceutical composition comprising voriconazole or pharmaceutically acceptable salt, solvate, ester, derivatives, hydrate, enantiomer, polymorph, prodrugs, complex or mixtures thereof wherein the said drug is dispersed in an aqueous, non-aqueous, or oily vehicle, or mixtures thereof.

According to third aspect of the present invention, there is provided a method of improving the stability of the pharmaceutical composition comprising voriconazole by dispersing the said drug or pharmaceutically acceptable salt, solvate, ester, derivatives, hydrate, enantiomer, polymorph, prodrugs, complex or mixtures thereof in an aqueous, non-aqueous, or oily vehicle, or mixtures thereof.

According to fourth aspect of the present invention, there is provided use of a pharmaceutical composition comprising voriconazole or pharmaceutically acceptable salt, solvate, ester, derivatives, hydrate, enantiomer, polymorph, prodrugs, complex or mixtures thereof dispersed in an aqueous, non-aqueous, or oily vehicle, or mixtures thereof, in the manufacture of a medicament for treating topical or systemic fungal infection in patients in need thereof.

According to fifth aspect of the present invention, there is provided a method of preventing or treating patients in need thereof comprising administering a ready-to-use pharmaceutical composition comprising voriconazole or pharmaceutically acceptable salt, solvate, ester, derivatives, hydrate, enantiomer, polymorph, prodrugs, complex or mixtures thereof dispersed in an aqueous, non-aqueous, or oily vehicle, or mixtures thereof.

### DETAILED DESCRIPTION

Various studies have been reported for preparing topical voriconazole formulation from the commercialized lyophilized product which is available for parenteral administration (Vfend® 200 mg IV, Pfizer) wherein lyophilized powder, is diluted with sodium chloride 0.9% under sterile conditions. (Preparation and Stability of Voriconazole Eye Drop Solution. Antimicrob, Agents Chemother. February 2009 vol. 53 no. 2 798-799).

The potential of Voriconazole for the treatment of ophthalmic diseases like keratomycosis have been reported by several authors. Nevertheless several studies have been reported wherein patients having ophthalmic diseases are being treated with lyophilized powder for injection formulation diluted with sodium chloride 0.9% under sterile conditions. However from the practicability aspect, such dilutions are not feasible at the consumer/patient level.

Further, due to poor stability of the molecule in aqueous vehicle and the various concerns as discussed above, there is an unmet need to develop a stable, ready-to-use composition of voriconazole.

The inventors of the present invention have surprisingly found that ready-to-use voriconazole compositions may be prepared by dispersing the drug in an aqueous, non-aqueous, or oily medium, or mixture thereof, without compromising the stability of the drug. Such compositions may also advantageously exhibit controlled impurity profiles.

The present invention thus provides a pharmaceutical composition comprising voriconazole and an aqueous, non-aqueous, or oily medium, or mixture thereof, and optionally one or more pharmaceutically acceptable excipients.

In one embodiment, the invention provides a pharmaceutical composition comprising voriconazole, an oily medium or mixture thereof, and optionally one or more pharmaceutically acceptable excipients.

In one embodiment, the invention provides a pharmaceutical composition comprising voriconazole, an oily medium or mixture thereof, a surfactant, and optionally one or more pharmaceutically acceptable excipients.

In one embodiment, the invention provides a pharmaceutical composition comprising voriconazole, an oily medium or mixture thereof, a surfactant, a pH adjusting agent, and optionally one or more pharmaceutically acceptable excipients.

In one embodiment, the invention provides a pharmaceutical composition comprising voriconazole, an aqueous medium, a surfactant, and optionally one or more pharmaceutically acceptable excipients.

In one embodiment, the invention provides a pharmaceutical composition comprising voriconazole, an aqueous medium, a surfactant, a pH adjusting agent, and optionally one or more pharmaceutically acceptable excipients.

In one embodiment, the invention provides a pharmaceutical composition comprising voriconazole, a non-aqueous medium, and optionally one or more pharmaceutically acceptable excipients.

In one embodiment, the invention provides a pharmaceutical composition comprising voriconazole, a non-aqueous medium, a surfactant and optionally one or more pharmaceutically acceptable excipients.

In one embodiment, the invention provides a pharmaceutical composition comprising voriconazole, a non-aqueous medium, a surfactant, a pH adjusting agent and optionally one or more pharmaceutically acceptable excipients.

Preferably, the pharmaceutical composition of the present invention is in ready-to-use form.

Within the scope of the present invention, reference to the term “voriconazole” is used throughout the description in broad sense to include not only the voriconazole per se but also pharmaceutically acceptable salts, solvates, esters, hydrates, enantiomers, derivatives, polymorphs and prodrugs thereof.

As used herein, the term “dispersed” shall include pharmaceutical compositions in which voriconazole is dispersed, suspended or dissolved in an aqueous, non-aqueous, or oily medium, or mixture thereof. The term “dispersing” shall be interpreted accordingly.

As used herein the term “vehicle”, “media” or “medium” are used interchangeably throughout the specification.

In one embodiment, the pharmaceutical composition of the present invention comprises an oily vehicle or mixture thereof. The oil or mixture of oils may comprise any pharmaceutically acceptable oil which is systemically or topically well tolerated.

Examples of oils suitable for use in a composition according to the present invention include, but are not limited to, castor oil, medium chain triglycerides (MCTs), mineral oils, vegetable oils, oily fatty acids, oily fatty alcohols, esters of sorbitol, fatty acids, oily sucrose esters, and any combination thereof. Examples of suitable vegetable oils include cotton seed oil, ground nut oil, corn oil, germ oil, olive oil, palm oil, soybean oil, sweet almond oil, sesame oil, and any combination thereof. Examples suitable of mineral oils include silicone oil, petrolatum oil, liquid paraffin and any combination thereof. Examples of suitable medium chain triglycerides include coconut oil; hydrogenated oils comprising hydrogenated cottonseed oil, hydrogenated palm oil, hydrogenated castor oil, hydrogenated soybean oil and any combination thereof. Preferably, the oily medium is liquid paraffin, castor oil, a medium chain triglyceride, or any combination thereof.

In another embodiment, the pharmaceutical composition of the present invention comprises a non-aqueous medium or mixture thereof. The non-aqueous medium, or mixture thereof, may comprise any pharmaceutically acceptable non-aqueous medium. Examples of non-aqueous vehicle suitable for use in a composition according to the present invention include, but are not limited to, glycerin, polyethylene glycol, propylene glycol, or any combination thereof.

In an alternative embodiment, the pharmaceutical composition of the present invention comprises an aqueous vehicle. In one embodiment, the pharmaceutical composition of the present invention comprises an aqueous vehicle and is substantially free from cyclodextrin or a derivative thereof.

The pharmaceutical composition of the present invention is in semi-solid or liquid form. Examples of suitable semi-solid forms include creams, ointments, lotions and the like. Examples of suitable liquid forms include dispersions, suspensions and solutions and the like.

In one embodiment, the pharmaceutical composition of the present invention is in a form that is suitable for topical or systemic administration.

The pharmaceutical composition of the invention for topical use may be formulated to administer directly to the eye or ear. The pharmaceutical composition may take the form of drops, a suspension, a nanosuspension, an ointment, a cream, a biodegradable dosage form such as an absorbable gel, a sponge, or collagen, a non-biodegradable dosage form such as (e.g. silicone) implants, wafers, lenses and particulate or vesicular systems, such as niosomes, emulsion, or a microemulsion and the like.

The pharmaceutical composition of the invention for systemic use may be formulated and administered parenterally via intravenous, intramuscular, subcutaneous, intraperitoneal, intrathecal routes of administration. The pharmaceutical composition may take the form of a suspension, a nanosuspension, or a particulate or vesicular system, such as niosomes, emulsion, liposomes, or a microemulsion and the like.

According to another embodiment, the pharmaceutical compositions of the invention may also be developed into dosage forms suitable to administer topically to the skin or mucosa, that is, dermally or transdermally. Typical formulations for this purpose may comprise gels, hydrogels, lotions, solutions, creams, ointments, dressings, foams, films, skin patches, wafers, implants, sponges, fibres, bandages and microemulsions.

Other means of topical administration include delivery by electroporation, iontophoresis, phonophoresis, sonophoresis and microneedle or needle-free (e.g. Powderject™, Bioject™, etc.) injection.

The pharmaceutical composition of the invention may also be administered intranasally or by inhalation, typically as an aerosol spray from a pressurized container or nebulizer, with or without the use of a suitable propellant, such as 1,1,1,2-tetrafluoroethane or 1,1,1,2,3,3,3-heptafluoropropane or mixtures thereof.

Compositions for inhaled/intranasal administration may be formulated to be immediate and/or modified release. Modified release formulations include delayed, sustained, pulsed, controlled, targeted and programmed release.

In addition to the aqueous, non-aqueous, or oily medium, or mixture thereof, the pharmaceutical composition of the present invention may comprise one or more additional pharmaceutically acceptable excipients. Examples of suitable pharmaceutically acceptable excipients include one or more polymers, wetting agents or surfactants, pH adjusting agents, isotonicity adjusting agents, preservatives, buffers, and chelating agents, or any combination thereof.

Examples of suitable pharmaceutically acceptable polymers include, but are not limited to, cellulose derivates (such as hydroxypropylcellulose, hydroxymethylcellulose, hydroxypropylmethylcellulose, methylcellulose polymers, hydroxyethylcellulose, sodium carboxymethylcellulose, carboxymethylene and carboxymethyl hydroxyethylcellulose or any combination thereof); and acrylics (such as acrylic acid, acrylamide, and maleic anhydride polymers, copolymers or their mixtures thereof) and mixtures thereof. Polymer blends may also be employed. A preferred pharmaceutically acceptable polymer is hydroxyethyl cellulose. In an embodiment, the pharmaceutically acceptable polymer is present in an amount from about 0.01% to about 5.0% (w/v), preferably from about 0.05% to about 2% (w/v), and more preferably from about 0.1% to about 1.0% (w/v), such as about 0.1, 0.2, 0.5, 1.0% (w/v).

Examples of suitable pharmaceutically acceptable wetting agents or surfactants include, but are not limited to, amphoteric, non-ionic, cationic or anionic molecules. Suitable surfactants include, but are not limited to, polysorbates, sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N,N-dimethyldodecylamine-N-, oxide, hexadecyltrimethylammonium bromide, polyoxyl 10 lauryl ether, Brij® surfactants (polyoxyethylene vegetable-based fatty ethers derived from lauryl, cetyl, stearyl and oleyl alcohols), bile salts (such as sodium deoxycholate and sodium cholate), polyoxyl castor oil, nonylphenol ethoxylate, cyclodextrins, lecithin, methylbenzethonium chloride, carboxylates, sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols (ethoxylated and sulphated), ethoxylated aliphatic alcohol, polyoxyethylene surfactants, carboxylic esters, polyethylene glycol esters, anhydrosorbitol ester and ethoxylated derivatives thereof, glycol esters of fatty acids, carboxylic amides, monoalkanolamine condensates, polyoxyethylene fatty acid amides, quaternary ammonium salts, amines with amide linkages, polyoxyethylene alkyl and alicyclic amines, N,N,N,N tetrakis substituted ethylenediamines, 2-alkyl 1-hydroxyethyl 2-imidazolines, N-coco 3-aminopropionic acid/sodium salt N-tallow 3-iminodipropionate disodium salt, N-carboxymethyl n dimethyl n-9 octadecenyl ammonium hydroxide, n-cocoamidethyl n-hydroxyethylglycine sodium salt and the like, polyoxyethylene, sorbitan monolaurate and stearate, Cremophor® (polyethoxylated castor oil), Solutol® (ethylene oxide/12-hydroxy stearic acid), polysorbate, tyloxapol and any combination thereof. Preferred pharmaceutically acceptable surfactants include tyloxapol and Span® 80 (sorbitane monooleate) or a mixture thereof. In an embodiment, the pharmaceutically acceptable wetting agent or surfactant is present in an amount from about 0.01% to about 5.0% (w/v), preferably from about 0.05% to about 2% (w/v), and more preferably from about 0.1% to about 1.0% (w/v), such as about 0.1, 0.2, 0.5, 1.0% (w/v).

Examples of suitable pharmaceutically acceptable isotonicity adjusting agents include, but are not limited to, D-mannitol, glucose, glycerol, sodium chloride, potassium chloride, calcium chloride and magnesium chloride, or any combination thereof. Various nitrates, citrates, acetates or mixtures thereof may also be employed. In an embodiment, the pharmaceutically acceptable isotonicity adjusting agents is present in an amount from about 0.1% to about 5.0% (w/v), preferably from about 1% to about 3% (w/v).

The pharmaceutical composition of voriconazole according to the present invention may comprise a suitable pharmaceutically acceptable pH adjusting agent, for example to adjust the pH of the composition suitable for topical or systemic administration. It would also be appreciated that pH of the pharmaceutical composition of the present invention can be modified based on the route of administration, dosage delivery form and particular patient need. For example, in the case of an ophthalmic composition, the pH of the composition is suitably adjusted between about pH 4 to 7.

Examples of suitable pharmaceutically acceptable pH adjusting agents include, but are not limited to, sodium hydroxide, citric acid, hydrochloric acid, boric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, or any combination thereof. In an embodiment, the pharmaceutically acceptable pH adjusting agent is present in an amount from about 0.01% to about 2.0% (w/v), preferably from about 0.05% to about 1% (w/v).

Examples of suitable pharmaceutically acceptable preservatives include, but are not limited to, benzalkonium chloride, benzethonium chloride and cetyl pyridinium chloride, benzyl bromide, benzyl alcohol, disodium EDTA, phenylmercury nitrate, phenylmercury acetate, thimerosal, merthiolate, acetate and phenylmercury borate, polymyxin B sulphate, chlorhexidine, methyl and propyl parabens, phenylethyl alcohol, quaternary ammonium chloride, sodium benzoate, sodium propionate, stabilized oxychloro complex, and sorbic acid or their mixtures thereof. Preferred pharmaceutically acceptable preservatives include disodium EDTA (edetate disodium) and benzalkonium chloride or a mixture thereof. In an embodiment, the pharmaceutically acceptable preservative is present in an amount from about 0.01% to about 2.0% (w/v), preferably from about 0.05% to about 1% (w/v).

Examples of suitable pharmaceutically acceptable buffers include, but are not limited to, sodium chloride, dextrose, lactose and phosphate buffered saline (PBS) or any combination thereof. Other suitable pharmaceutically acceptable buffers include, but are not limited to, disodium succinate hexahydrate, borate, citrate, phosphate, acetate, physiological saline, tris-HCl(tris-(hydroxymethyl)-aminomethane hydrochloride), HEPES (N-2-hydroxyethyl piperazine-N1-2-ethane sulfonic acid), sodium phosphate, sodium borate, physiological saline, citrate, carbonate, phosphate and/or mixtures thereof to achieve the desired osmolarity. In an embodiment, the pharmaceutically acceptable buffer is present in an amount from about 0.01% to about 2.0% (w/v), preferably from about 0.05% to about 1% (w/v).

Examples of suitable pharmaceutically acceptable chelating agents include, but are not limited to, ethylenediaminetetraacetic acid (EDTA), disodium EDTA and derivatives thereof, citric acid and derivatives thereof, niacinamide and derivatives thereof, and sodium deoxycholate and derivatives thereof or mixtures of chelating agents thereof. In an embodiment, the pharmaceutically acceptable chelating agent is present in an amount from about 0.01% to about 2.0% (w/v), preferably from about 0.05% to about 1% (w/v).

In a preferred embodiment, there is provided a pharmaceutical composition comprising voriconazole, an aqueous medium and one or more wetting agents, preferably tyloxapol. The composition may further comprise one or more polymers, pH adjusting agents, isotonicity adjusting agents, preservatives, buffers, and chelating agents, or any combination thereof, of the types described herein.

In a further preferred embodiment, there is provided a pharmaceutical composition comprising voriconazole, an oily medium; preferably liquid paraffin, a medium chain triglyceride and/or castor oil, and one or more wetting agents, preferably tyloxapol. The composition may further comprise one or more polymers, pH adjusting agents, isotonicity adjusting agents, preservatives, buffers, and chelating agents, or any combination thereof, as described herein.

In a further preferred embodiment, there is provided a pharmaceutical composition comprising voriconazole, an oily vehicle, preferably liquid paraffin, and a preservative, preferably benzalkonium chloride.

It will be appreciated that the precise therapeutic dose of voriconazole will depend on the age and condition of the patient and the nature of the condition to be treated and will be at the ultimate discretion of the physician. In an embodiment, the pharmaceutical composition of the invention comprises between about 50 mg to about 200 mg of voriconazole, such as 50, 100, 150 or 200 mg.

The present invention also provides processes for preparing stable pharmaceutical compositions comprising voriconazole.

In one embodiment, there is provided a process for preparing a pharmaceutical composition comprising voriconazole, which process comprises dispersing, suspending or dissolving voriconazole in an aqueous, non-aqueous, or oily medium, or a mixture thereof. Preferably, the pharmaceutical composition is a ready-to-use composition. Preferably, the medium is an oil, or mixture thereof.

According to one embodiment, the present invention provides a process of preparing a pharmaceutical composition comprising voriconazole, which process comprises the steps of: (a) dissolving one or more of a chelating agent, buffering agent, isotonicity agent and/or preservative in a suitable aqueous medium, such as water for injection; (b) milling voriconazole in the presence of one or more surfactants; (c) adding the milled drug to the product of step (a); (d). preparing a separate mixture of a suitable polymer such as hydroxyethyl cellulose and an aqueous medium, and autoclaving the mixture; (e) adding the drug mixture obtained in step (c) to polymer mixture obtained in step (d); and optionally (f) making the volume with water for injection and adjusting the pH.

According to another embodiment, the present invention provides a process of preparing a pharmaceutical composition comprising voriconazole, which process comprises the steps of: (a) dispersing, suspending or dissolving voriconazole in a mixture of one or more oils and one or more surfactants; (b) adding a suitable preservative such as benzalkonium chloride to the drug-containing mixture; and optionally (c) adding additional oil to make up the final volume.

According to yet another embodiment, the present invention provides a process of preparing a pharmaceutical composition comprising voriconazole, which process comprises the steps of: (a) dispersing, suspending or dissolving voriconazole and a suitable preservative such as benzalkonium chloride in one or more oils; and optionally (b) adding additional oil to make up the final volume. The present invention also provides a method of preventing or treating a topical or systemic fungal infection comprising administering a ready-to-use pharmaceutical composition comprising voriconazole to a patient in need thereof.

Further, the present invention also provides use of a ready-to-use pharmaceutical composition comprising voriconazole in the manufacture of a medicament for treating topical or systemic fungal infection.

The following examples are for the purpose of illustration of the invention only and are not intended in any way to limit the scope of the present invention.

### Example 1

### Suspension Formulation

| | | Quantity |
|---|---|---|
| Sr. no. | Ingredients | (% w/v) |
| 1. | Voriconazole | 0.3 |
| 2. | Tyloxapol | 0.05 |
| 3. | Boric acid | 0.6 |
| 4. | Sodium chloride | 0.53 |
| 5. | Edetate disodium | 0.01 |
| 6. | Benzalkonium chloride | 0.01 |
| 7. | Hydroxyethyl cellulose | 0.2 |
| 8. | Sodium hydroxide/Hydrochloric acid | q.s. pH 5.5-7.5 |
| 9. | Water for injection | q.s. to 100 ml |

1. Tyloxapol was solubilized in water with the aid of heat. Drug was added to this solution followed by autoclave at 121° C. for 30 min. Mixture was cooled and then ball milled.
2. Hydroxyethylcellulose was added to water and heated.
3. Edetate disodium, Boric acid, Sodium Chloride, and Benzalkonium chloride were added to water and filtered, followed by addition of the drug part (1).
4. Step 3 mixture was added to step 2, final volume was made up with water and pH was adjusted.

### Example 2

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Span 80 | 0.1 |
| 4. | Liquid Paraffin | q.s. to 100 ml |

### Process:

1. Voriconazole was dispersed in span 80 and part of the liquid paraffin added under stirring, followed by addition of Benzalkonium chloride.
2. Final volume was made up with liquid paraffin.

### Example 3

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Span 80 | 0.1 |
| 4. | Medium chain triglyceride | q.s. to 100 ml |

Process:
1. Voriconazole was dispersed in span 80 and part of the medium chain triglyceride added under stirring, followed by addition of Benzalkonium chloride.
2. Final volume was made up with medium chain triglyceride.

### Example 4

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Span 80 | 1.0 |
| 4. | Castor oil | q.s. to 100 ml |

### Process:

1. Voriconazole was dispersed in span 80 and part of the castor oil added under stirring, followed by addition of Benzalkonium chloride.
2. Final volume was made up with castor oil.

### Example 5

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Liquid Paraffin | q.s. to 100 ml |

### Process:

1. Voriconazole was dispersed in part of the liquid paraffin under stirring followed by addition of Benzalkonium chloride.
2. Final volume was made up with liquid paraffin.

### Example 6

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Medium chain triglyceride | q.s. to 100 ml |

### Process:

1. Voriconazole was dispersed in part of the medium chain triglyceride under stirring followed by addition of Benzalkonium chloride.
2. Final volume was made up with medium chain triglyceride.

### Example 7

### Oily Formulation

| | | Quantity |
|---|---|---|
| Sr. no. | Ingredients | (% w/v) |
| 1. | Voriconazole | 0.3 |
| 2. | Benzalkonium chloride | 0.01 |
| 3. | Medium chain triglyceride | 50 |
| 4. | Liquid Paraffin | 49.69 |

### Process:

1. Voriconazole was dispersed in part of the medium chain triglyceride and liquid paraffin under stirring followed by addition of Benzalkonium chloride.
2. Final volume was made up with medium chain triglyceride and liquid paraffin.

TABLE 1
Stability study
The below stability data illustrates the stability
of the composition of the present invention.

| | Condition parameters | | | |
|---|---|---|---|---|
| | | 25° C./60% | 30° C./65% | 40° C./20% |
| | Initial | RH after 3 M | RH after 3 M | RH after 3 M |
| Assay (%) | 100.1 | 100.2 | 100.5 | 100.3 |
| Single max | 0.020 | 0.019 | 0.016 | 0.027 |
| impurity | | | | |
| Total imp | 0.061 | 0.042 | 0.049 | 0.081 |

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a polymer” includes a single polymer as well as two or more different polymers; reference to a “plasticizer” refers to a single plasticizer or to combinations of two or more plasticizer, and the like.

1. An ophthalmic pharmaceutical composition comprising voriconazole and optionally one or more pharmaceutically acceptable excipients.

2. An ophthalmic pharmaceutical composition as claimed in claim 1 comprising voriconazole, a vehicle and optionally one or more pharmaceutically acceptable excipients.

3. An ophthalmic pharmaceutical composition as claimed in claim 1 wherein the vehicle is oily, aqueous or non aqueous or mixtures thereof.

4. An ophthalmic pharmaceutical composition according to claim 1, wherein voriconazole is present in the form of as a pharmaceutically acceptable salt, pharmaceutically acceptable solvate, pharmaceutically acceptable hydrate, pharmaceutically acceptable ester, pharmaceutically acceptable enantiomer, pharmaceutically acceptable derivative, pharmaceutically acceptable polymorph, pharmaceutically acceptable prodrug, or pharmaceutically acceptable complex thereof.

5. An ophthalmic pharmaceutical composition according to claim 1, comprising voriconazole, an oily vehicle, or mixture thereof, and optionally one or more pharmaceutically acceptable excipients.

6. An ophthalmic pharmaceutical composition according to claim 1, wherein the oily vehicle comprises castor oil, liquid paraffin, medium chain triglycerides, mineral oils, vegetable oils, oily fatty acids, oily fatty alcohols, esters of sorbitol, fatty acids, oily sucrose esters, or any combination thereof.

7. An ophthalmic pharmaceutical composition according to claim 1, wherein the non-aqueous vehicle comprises glycerin, polyethylene glycol, propylene glycol, or any combination thereof.

8. An ophthalmic pharmaceutical composition according to claim 1 in ready-to-use form.

9. An ophthalmic pharmaceutical composition according to claim 1, comprising one or more polymers, surfactants or wetting agents, pH adjusting agents, isotonicity adjusting agents, preservatives, buffers, chelating agents, or any combination thereof.

10. An ophthalmic pharmaceutical composition according to claim 9, wherein the polymer comprises hydroxypropylcellulose, hydroxymethylcellulose, hydroxypropylmethylcellulose, hydroxyethylcellulose, sodium carboxymethylcellulose, carboxymethylene, carboxymethyl hydroxyethylcellulose, acrylic acid, acrylamide, maleic anhydride polymers and copolymers, or any combination thereof.

11. An ophthalmic pharmaceutical composition according to claim 9, wherein the surfactant or wetting agent comprises polysorbates, sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N, N-dimethyldodecylamine-N-oxide, hexadecyltrimethylammonium bromide, polyoxyl 10 lauryl ether, Brij® surfactants (polyoxyethylene vegetable-based fatty ethers derived from lauryl, cetyl, stearyl and oleyl alcohols), bile salts, polyoxyl castor oil, nonylphenol ethoxylate, cyclodextrins, lecithin, methylbenzethonium chloride, carboxylates, sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols (ethoxylated and sulphated), ethoxylated aliphatic alcohol, polyoxyethylene surfactants, carboxylic esters, polyethylene glycol esters, anhydrosorbitol ester and ethoxylated derivatives thereof, glycol esters of fatty acids, carboxylic amides, monoalkanolamine condensates, polyoxyethylene fatty acid amides, quaternary ammonium salts, amines with amide linkages, polyoxyethylene alkyl and alicyclic amines, N,N,N,N tetrakis substituted ethylenediamines, 2-alkyl 1-hydroxyethyl 2-imidazolines, N-coco 3-aminopropionic acid/sodium salt N-tallow 3-iminodipropionate disodium salt, N-carboxymethyl n dimethyl n-9 octadecenyl ammonium hydroxide, n-cocoamidethyl n-hydroxyethylglycine sodium salt and the like, polyoxyethylene, sorbitan monolaurate and stearate, Cremophor® (polyethoxylated castor oil), Solutol® (ethylene oxide/12-hydroxy stearic acid), polysorbate, tyloxapol, or any combination thereof.

12. An ophthalmic pharmaceutical composition according to claim 9, wherein the isotonicity adjusting agent comprises D-mannitol, glucose, glycerol, sodium chloride, potassium chloride, calcium chloride, magnesium chloride, or any combination thereof.

13. An ophthalmic pharmaceutical composition according to claim 9, wherein the pH adjusting agent comprises sodium hydroxide, citric acid, hydrochloric acid, boric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, or any combination thereof.

14. An ophthalmic pharmaceutical composition according to claim 9, wherein the preservative comprises benzalkonium chloride, benzethonium chloride and cetyl pyridinium chloride, benzyl bromide, benzyl alcohol, disodium EDTA, phenylmercury nitrate, phenylmercury acetate, thimerosal, merthiolate, acetate and phenylmercury borate, polymyxin B sulphate, chlorhexidine, methyl and propyl parabens, phenylethyl alcohol, quaternary ammonium chloride, sodium benzoate, sodium propionate, stabilized oxychloro complex, sorbic acid, or any combination thereof.

15. An ophthalmic pharmaceutical composition according to claim 9, wherein the buffer comprises sodium chloride, dextrose, lactose and phosphate buffered saline (PBS), disodium succinate hexahydrate, borate, citrate, phosphate, acetate, physiological saline, tris-HCl(tris-(hydroxymethyl)-aminomethane hydrochloride), HEPES (N-2-hydroxyethyl piperazine-N1-2-ethane sulfonic acid), sodium phosphate, sodium borate, physiological saline, citrate, carbonate, phosphate and/or mixtures thereof.

16. An ophthalmic pharmaceutical composition according to claim 9, wherein the chelating agent comprises ethylenediaminetetraacetic acid (EDTA), disodium EDTA and derivatives thereof, citric acid and derivatives thereof, niacinamide and derivatives thereof, and sodium deoxycholate and derivatives thereof, or any combination thereof.

17. An ophthalmic pharmaceutical composition according to claim 1 in the form of a cream, ointment, lotion, dispersion, suspension, solution, drop, gel, emulsion or microemulsion.

18. A process for preparing an ophthalmic pharmaceutical composition according to claim 1, said process comprising dispersing voriconazole in an aqueous, non-aqueous or oily vehicle or mixture thereof.

19. A process according to claim 18, comprising the steps of: (a) dispersing, voriconazole in one or more oily vehicle and optionally one or more surfactants; (b) adding a preservative to step (a) and (c) making up the final volume with the oily vehicle.

20. A process according to claim 19, comprising the steps of: (a) dispersing voriconazole and a preservative in one or more oily vehicle; and (c) making up the final volume with the oily vehicle.

21. Use of an ophthalmic pharmaceutical composition according to claim 1 in the manufacture of a medicament for the prevention or treatment of a fungal infection.

22. A method for the prevention or treatment of a fungal infection comprising administering to a patient in need thereof a pharmaceutical composition according to claim 1.

23. An ophthalmic pharmaceutical composition according to claim 1 for use in the prevention or treatment of a fungal infection.
